(19) United States
(12) Patent Application Publication
(10) Pub. No.: US 2025/0012798 A1
(43) Pub. Date: Jan. 9, 2025

(54) METHODS FOR DEFINING STAGES AND PROGRESSION OF AMYOTROPHIC LATERAL SCLEROSIS

(71) Applicant: The General Hospital Corporation, Boston, MA (US)

(72) Inventors: Susan Raju PAUL, Kochi (IN); Mark POZNANSKY, Newton, MA (US); Ruxandra F. SIRBULESCU, Brighton, MA (US); James D. BERRY, Belmont, MA (US)

(21) Appl. No.: 18/707,478

(22) PCT Filed: Nov. 14, 2022
(86) PCT No.: PCT/US2022/049847

§ 371 Date: May 3, 2024

Related U.S. Application Data
(60) Provisional application No. 63/278,114, filed on Nov. 11, 2021.

(51) Int. Cl. G01N 33/569; A61K 31/4152; A61K 31/428; A61K 45/06; G01N 15/01

(52) U.S. Cl. CPC G01N 33/56972; A61K 31/4152; A61K 31/428; A61K 45/06; G01N 15/01; G01N 2015/016; G01N 2333/70589

(57) ABSTRACT

The invention features a method including characterizing a white blood cell sample from a patient using cytometry (e.g., CyTOF); wherein a deficiency in regulatory or suppressive immune cells and increased activated immune cells in the sample, relative to a healthy sample, indicates that the patient has amyotrophic lateral sclerosis (ALS).

### CLAIM OF PRIORITY

This application claims benefit of U.S. Provisional Patent Application Ser. No. 63/278,114 filed Nov. 11, 2021. The entire contents of the foregoing are hereby incorporated by reference.

### BACKGROUND OF THE INVENTION

The present invention relates to methods for diagnosis of amyotrophic lateral sclerosis (ALS) and for monitoring ALS progression, as well as to methods for treatment of the disease.

Amyotrophic lateral sclerosis, or ALS, is a progressive nervous system disease that affects nerve cells in the brain and spinal cord, causing loss of muscle control. ALS often begins with muscle twitching and weakness in a limb, or slurred speech. Eventually, ALS affects control of the muscles needed to move, speak, eat and breathe. There is no cure—the disease is fatal. ALS affects the nerve cells that control voluntary muscle movements such as walking and talking (motor neurons). ALS causes the motor neurons to gradually deteriorate, and then die. Motor neurons extend from the brain to the spinal cord to muscles throughout the body. When motor neurons are damaged, they stop sending messages to the muscles, so the muscles do not function. In 5-10% of cases the condition is inherited; in the rest, the cause is unknown but may involve a complex interaction between genetic and environmental factors (called “sporadic ALS”). Early diagnosis is typically difficult because ALS can mimic other neurological diseases. Currently, tests to rule out other neurological conditions may include an electromyogram to detect abnormalities in the electrical activity in muscle contraction and release, nerve conduction studies that measure the ability of the nerves to send impulses, magnetic resonance imaging (MRI) imaging of the brain and spinal cord to eliminate conditions caused by spinal cord tumors and herniated disks, blood and urine tests for other diseases and conditions, and muscle biopsies to eliminate muscle diseases. There is a need in the art for methods which identify stages and progression of ALS.

### SUMMARY OF THE INVENTION

This invention provides methods and materials involved in assessing immune system profiles relating to amyotrophic lateral sclerosis (ALS). For example, this document provides methods and materials for performing flow cytometry to determine the immune status of a patient (e.g., a human) using a white blood sample to determine the number of leukocyte subsets in circulation. In some cases, the immune status of a patient is determined by measuring, for example, the number of CD4+ lymphocytes, CD8+ lymphocytes, regulatory T cells, B cells, NK cells, granulocytes, etc as is disclosed herein). The immune status can be determined by quantitating representatives of each major category of leukocytes (e.g., granulocytes, NK cells, T cells, B cells, lymphocytes etc. as is disclosed herein).

Accordingly, the present invention relates to the use of immune-cell proteomic signatures to define stages and progression of ALS and to inform clinical decision-making regarding inter alia diagnosis, therapeutic targeting, choice of therapy, treatment efficacy monitoring, and prognosis.

The invention, in general, features a method including characterizing a white blood cell sample from a patient using cytometry (e.g., CyTOF); wherein a deficiency in regulatory or suppressive immune cells and increased activated immune cells in the sample, relative to a healthy sample, indicates that the patient has amyotrophic lateral sclerosis (ALS).

In embodiments, the sample is incubated with antibodies that specifically bind granulocytes, monocytes, dendritic cells, T cells, B cells, NK cells, immune activating cells, or immune suppressive cells. In embodiment, the regulatory or suppressive immune cells include Treg, Breg, or M2 macrophage clusters. In embodiments, the activated immune cells include T and B effector and NK effector cell clusters.

In embodiments, the method includes calculating numbers of immune cells and proportion of the total leukocyte population of the sample using a pan human leukocyte marker (e.g. CD45).

In embodiments, cytometry is cell or mass cytometry.

In embodiments, the method includes performing total RNA sequencing on the sample to delineate subpopulations of leukocyte populations and TCR and BCR expression analysis, viral genome analysis and/or HLA analysis.

In embodiments. The method includes including identifying clusters of leukocytes in the sample.

In embodiments, identifying includes cluster analysis, linear regression analysis, linear discrimination analysis and/or elastic net logistical analysis. In embodiments, the clusters of leukocytes segregate between healthy individuals and individuals with ALS (such as late ALS or early ALS).

In embodiments, the patient has a deficiency in Treg, Breg, or M2 macrophage clusters.

In embodiments, the patient has increased activated immune cell cluster T and B effectors.

In embodiments, the patient has increased activated NK effector clusters.

In embodiments, the method further includes administering to the patient a therapy for treating ALS. In embodiments, the therapy is riluzole or edarvarone.

In embodiments, FoxP3+ B regulatory cells have lower abundance in the ALS patient.

In embodiments, mature B cells including CD11c expression are increased in patients with a lower ALSFRS-R as compared to higher ALSFRS-R and healthy controls.

In embodiments, CD4 T cells are increased in the ALS patient.

In embodiments, CD8 T cells are increased in the ALS patient.

In embodiments, activated CD4 T cells are elevated in an ALS patient having a lower ALSFRS-R as compared to patients with higher ALSFRS-R and healthy controls.

In embodiments, CD11c+ monocytes are increased in the ALS patient.

In embodiments, NK T cells are increased in the ALS patient.

In embodiments, activated B cells (CD19+ CD20+ IgD+ IgM+) are decreased in the ALS patient,

In embodiments, memory B cells (CD19+ CD20+ CD21+ CD27+) are decreased in the ALS patient.

In embodiments, activated CD4 T cells (CD27+ PD1+) are increased in the ALS patient.

In embodiments, CD8 T cells (CD27+ CD7+) are increased in the ALS patient.

In embodiments, CD4 T cells (CD25+ CD27+ CD39+) are increased in ALS patients with lower ALSFRS-R as compared to ALS patients with higher ALSFRS-R and healthy controls.

In embodiments, NK T cells are increased in ALS patients.

In embodiments, the method includes determining a level of at least 2, 3, 4, 5, 6, 7, 8, 9, 10, 11, 12 or more markers as listed in FIG. 2, FIG. 8B, FIG. 10B, FIG. 12, FIG. 14B, and FIG. 15B.

In embodiments, the sample includes a phenotype as depicted in cluster 387, 392, 394, 408, or 422.

In embodiments, the sample includes a phenotype as depicted in cluster 951, 947, 961, 953, 945, 955, 954, 949, 944, 956, or 962.

In embodiments, the sample includes a phenotype as depicted in cluster 21, 28, 44, 92, or 37.

In another aspect, the invention features a method including:
(a) determining whether a patient has ALS according to any of the aforementioned methods;
(b) analyzing a second white blood cell sample from the patient according to any of the aforementioned methods; and
(c) determining a deficiency in regulatory or suppressive immune cells and increased activated immune cells in the second sample.

In embodiments, the patient has an increased deficiency in Treg, Breg, or M2 macrophage clusters in the second sample compared to the sample taken at an earlier time point.

In embodiments, the patient has increased activated immune cell cluster T and B effectors in the second sample compared to the earlier monitored sample.

In embodiments, the patient has increased activated NK effector clusters in the second sample compared to compared to the earlier monitored sample.

In embodiments, the method further including administering to the patient a therapy for treating ALS.

In embodiments, the therapy includes immune cell therapy.

In embodiments, immune therapy includes administering B cells or Treg cells.

In embodiments, the therapy includes administering an immune modulating agent.

In embodiments, the immune modulating agent is Baracitinib or a Jak-Stat inhibitor.

In embodiments, the patient is experiencing a clinically meaningful decline from baseline in an ALSFRS-R total score at the time the second sample is obtained.

In embodiments, there are at least 2, 4, 6, 8, 10, 12, or 14 weeks between obtaining the first sample and the second sample.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, sequences, and other references mentioned herein are incorporated by reference in their entirety. In case of conflict, the present specification, including definitions, will control.

The invention provides several advantages and addresses an unmet need in ALS patient care. Current biomarkers for documenting or monitoring ALS progression rely on subjective or at best semi-quantitative clinical assessments (ALS-FRS scores) or blood markers including neurofilament measurements which so far have not been validated to predicting prognosis, specific clinical outcomes and/or treatment indicators. No single biomarkers derived from serum, plasma or cellular components of the blood, cerebrospinal fluid (CSF) or other body tissues have been defined that support quantitative monitoring of the patients' clinical progression with ALS, define the impact of standard of care or experimental therapies and support clinical decisions to start specific standard of care or experimental immune modulatory or anti-inflammatory therapies. The methods and approaches described herein are not only relevant to diagnosis of ALS but also to other standard clinical/neurological findings.

To date in ALS, immune signatures have not been identified that track with disease progression. While specific single immune cell populations (such as activated T cells) have been examined as biomarkers, immune signatures involving combinatorial and simultaneous assessments of B, T, NK cells, monocytes and neutrophils have not been defined. The methods described herein include focusing on evaluating B cell populations in depth in the context of ALS. Highly specific subpopulations of B cells and T cells which are differentially expressed between healthy controls and patients with ALS have been identified, as well as between healthy, high ALSFRS-R (Amyotrophic Lateral Sclerosis Functional Rating Scale) and low ALSFRS-R. These signature differences detailed herein delineate a collection of markers of disease progression that informs diagnosis of ALS, determination of the stage of the disease and assessment of the effect of standard of care therapies such as riluzole and edarvarone along with experimental therapies and therapies in clinical trials to assess the safety and efficacy of these agents. Presently there are no biomarkers in ALS that are used for this purpose.

Furthermore, as is described herein, surprisingly, CD11c expression is a distinguishing marker for B cells in ALS. CD11c+ B cells produce IL-10, a regulatory cytokine, even under optimal conditions. CD39 is an ectonucleotidase which is the rate-limiting enzyme in the conversion of ATP to immunomodulatory adenosine. In addition, CD39+ B cell subsets unexpectedly distinguished ALS patients from healthy controls.

Other features and advantages of the invention will be apparent from the following description of the preferred embodiments thereof, and from the claims.

### DESCRIPTION OF THE DRAWINGS

The present disclosure contains at least one drawing executed in color. Copies of this patent or patent application publication with color drawing(s) will be provided by the Office upon request and payment of the necessary fee.

FIG. 1 shows a study design involving 30 ALS and 15 age/gender matched controls.

FIG. 2 shows a CyTOF antibody panel.

FIG. 3 shows data analysis for a force directed layout of live cell clusters generated by X-shift clustering of an ALS data set. Each color indicates a different cluster. The distance between the clusters indicates their similarity.

FIG. 4A, FIG. 4B, FIG. 4C, and FIG. 40 show differential abundance of CD45+ cells in an unpaired t test. Volcano plots of CD45+ cell clusters are based on absolute abundances using a student unpaired t-test. The dotted red lines indicate p-value (y-axis) and fold change (x-axis) thresholds; to be significant, a cluster must be above both dotted lines. The size of the circle representing each cluster on the plot indicates the number of cells in that cluster. Although two clusters were identified through this unpaired t-test as being differentially abundant, both cluster 416 and cluster 396 are less than 0.1% of live cells and hence eliminated from downstream analysis.

FIG. 5 shows results for an elastic-net logistical regression of CD45+ cells. Elastic net waterfall plot of the clusters (y-axis) with the top 30 regression coefficients (x-axis). Coefficients represent the change in log odds per unit increase. In the training model used, there is an increased risk of the outcome as x increases. This can be transformed into an odds ratio using (exp(x-value))*100%, the percentage indicates that there is an increase (>100%), or decrease (<100%) in the odds of the outcome for each unit increase in x. Within the training model used here, the graph indicates that the clusters in grey would have a higher probability of having a greater abundance in the healthy controls, while the clusters in black have a higher probability of having a greater abundance in ALS patients. A (*) indicates the clusters which are lesser than 0.1% of CD45+ cells. Due to their small size, these clusters were not evaluated further.

FIG. 6 shows results of a phenotypic profile of CD45+ cell clusters identified through elastic-net logistic regression.

FIG. 7 shows results of a linear discriminant analysis: CD45+ healthy cells (n=6), ALS FRS29-39 (n=5) and ALS FRS40-47 (n=7). Linear discriminant analysis (LDA) models were constructed to rank combinations of up to 3 clusters based on the ability to distinguish healthy controls (N=6) from ALS FRS29-39 (N=5) and subsequently assess the distribution of ALS FRS40-47 (N=7). The discriminant functions with high predictive accuracy were used to project the three groups onto corresponding feature space and identify combinations of cell populations where the ALS FRS40-47 trend towards the healthy controls. LDA plots depict the distribution of each group, where the center of the x-axis denotes the point where the ALS FRS29-39 and healthy controls are maximally separated, and the y-axis reflects the count (or density estimate). The title of each plot indicates the clusters in the model. The accuracy of each model in indicated within the plot.

FIG. 8A and FIG. 8B show clusters identified by linear discriminant analysis of CD45+ cells. FIG. 8A shows scatter plots comparing healthy and ALS, and healthy, FRS 40-47 and FRS 29-29. The x-axis indicates the group and y-axis indicates the frequency of the cluster in CD45+ cells from each donor. The horizontal line represents the mean, and error bars indicate with the standard deviation. FIG. 8B shows a parallel coordinate plot representing the phenotype of cluster 422. Along the x-axis are the markers on which the clusters were defined, and the y-axis represents the intensity of expression of each marker in the clusters. The red line represents cluster 422, and the grey lines indicate the expression of other clusters in the study. Peaks represent the highest expression of that marker. Out of the total 499480 cells that were clustered, 101894 cells make up this cluster which is 4 20.4% of CD45+ cells that were clustered.

FIG. 9A, FIG. 9B, FIG. 9C, and FIG. 9D show differential abundance of B cells in an unpaired t test. Volcano Plot of B cell clusters are based on absolute abundances using a student unpaired t-test. The dotted red lines indicate p-value (y-axis) and fold change (x-axis) thresholds; to be significant, a cluster must be above both dotted lines. The size of the circle representing each cluster on the plot indicates the number of cells in that cluster.

FIG. 10A and FIG. 10B show differential abundance of B cells. Cluster 962 is significantly enriched in patients with FRS over 40. FIG. 10A shows scatter plots comparing healthy and ALS, and healthy, FRS 40-47 and FRS 29-29. x-axis indicates the group and y-axis indicates the frequency of the cluster in B cells from each donor. Horizontal line represents the mean, and error bars indicate with the standard deviation. FIG. 10B shows a parallel coordinate plot representing the phenotype of cluster 962. Along the x-axis are the markers on which the clusters were defined, and the y-axis represents the intensity of expression of each marker in the clusters. The red line represents cluster 962, and the grey lines indicate the expression of other clusters in the study. Peaks represent the highest expression of that marker. Out of the total 27545 cells that were clustered, 1212 cells make up this cluster which is 4.4% of B cells that were clustered. Based on expressions the cluster is CD11b^{low} CD11c^{low} CD21⁺ CD24+ CD27⁺ CD39^{mid} FoxP3⁺ HLADR⁺ IgG+.

FIG. 11 shows results of an elastic-net logistical regression of B cell clusters. FIG. 11 shows an elastic net waterfall plot of the clusters (y-axis) with the top 30 regression coefficients (x-axis). Coefficients represent the change in log odds per unit increase. In the training model used, there is an increased risk of the outcome as x increases. This can be transformed into an odds ratio using (exp(x-value))*100%, the percentage indicates that there is an increase (>100%), or decrease (<100%) in the odds of the outcome for each unit increase in x. Within the training model used here, the graph indicates that the clusters in grey would have a higher probability of having a greater abundance in the healthy controls, while the clusters in black have a higher probability of having a greater abundance in ALS patients. A (*) indicates the clusters which are lesser than 0.5% of B cells. Due to their small size, these clusters were not evaluated further.

FIG. 12 shows results of a phenotypic profile of B Cell Clusters identified through elastic-net logistic regression.

FIG. 13 shows results of a linear discriminant analysis: healthy B cells (n=6), ALS FRS29-39 (n=5) and ALS FRS40-47 (n=7). Linear discriminant analysis (LDA) models were constructed to rank combinations of up to 3 clusters based on the ability to distinguish healthy controls (N=6) from ALS FRS29-39 (N=5) and subsequently assess the distribution of ALS FRS40-47 (N=7). The discriminant functions with high predictive accuracy were used to project the three groups onto corresponding feature space and identify combinations of cell populations where the ALS FRS40-47 trend towards the healthy controls. LDA plots depict the distribution of each group, where the center of the x-axis denotes the point where the ALS FRS29-39 and healthy controls are maximally separated, and the y-axis reflects the count (or density estimate). The title of each plot indicates the clusters in the model. The accuracy of this model for B cells clusters was low as indicated in the table.

FIG. 14A and FIG. 14B show results of clusters identified by linear discriminant analysis of B cells. FIG. 14A shows scatter plots comparing healthy and ALS, and healthy, FRS 40-47 and FRS 29-29. x-axis indicates the group and y-axis indicates the frequency of the cluster in B cells from each donor. Horizontal line represents the mean, and error bars indicate with the standard deviation. FIG. 14B shows a parallel coordinate plot representing the phenotype of cluster 955. Along the x-axis are the markers on which the clusters were defined, and the y-axis represents the intensity of expression of each marker in the clusters. The red line represents cluster 955, and the grey lines indicate the expression of other clusters in the study. Peaks represent the highest expression of that marker. Out of the total 27545 cells that were clustered, 1333 cells make up this cluster which is 4.85% of B cells that were clustered. Based on expressions the cluster is CD11c+ CD39^{mid} HLADR+ IgD+ IgM+.

FIG. 15A and FIG. 15B show results of clusters identified by linear discriminant analysis of B cells. FIG. 15A shows scatter plots comparing healthy and ALS, and healthy, FRS 40-47 and FRS 29-29. The x-axis indicates the group and the y-axis indicates the frequency of the cluster in B cells from each donor. Horizontal line represents the mean, and error bars indicate with the standard deviation. FIG. 15B shows a parallel coordinate plot representing the phenotype of cluster 960. Along the x-axis are the markers on which the clusters were defined, and the y-axis represents the intensity of expression of each marker in the clusters. The red line represents cluster 960, and the grey lines indicate the expression of other clusters in the study. Peaks represent the highest expression of that marker. Out of the total 27545 cells that were clustered, 5741 cells make up this cluster which is 20.9% of B cells that were clustered. Based on expressions the cluster is CD21+ CD24+ CD25+ CD27+ CD39^{mid} CD69+ FoxP3^{low} HLADR+ IgD+ IgM+.

FIG. 16A and FIG. 16B show results of differential abundance of CD45+ cells in an unpaired t test. Volcano Plot of CD45+ cell clusters based on absolute abundances using a Student unpaired t-test. The dotted red lines indicate p-value (y-axis) and fold change (x-axis) thresholds; to be significant, a cluster must be above both dotted lines. The size of the circle representing each cluster on the plot indicates the number of cells in that cluster.

FIG. 17 shows results of an elastic-net logistical regression of CD45+ cell clusters. Elastic net waterfall plot of the clusters (y-axis) with regression coefficients (x-axis). Coefficients represent the change in log odds per unit increase. In the training model used, there is an increased risk of the outcome as x increases. This can be transformed into an odds ratio using (exp(x-value))*100%, the percentage indicates that there is an increase (>100%), or decrease (<100%) in the odds of the outcome for each unit increase in x. Within the training model used here, the graph indicates that the clusters in grey would have a higher probability of having a greater abundance in the healthy controls, while the clusters in black have a higher probability of having a greater abundance in ALS patients.

FIG. 18A and FIG. 185 show results of an elastic-net logistical regression of CD45+ cell clusters in which cluster>0.1% of total CD45+ cells.

FIG. 19 shows results of linear discriminant analysis: CD45+ healthy cells (n=11), ALS FRS21-39 (n=17) and ALS FRS40-47 (n=11). Linear discriminant analysis (LDA) models were constructed to rank combinations of up to 3 clusters based on the ability to distinguish healthy controls (N=11) from ALS FRS21-39 (N=17) and subsequently assess the distribution of ALS FRS40-47 (N=11). The discriminant functions with high predictive accuracy were used to project the three groups onto corresponding feature space and identify combinations of cell populations where the ALS FRS40-47 trend towards the healthy controls. LDA plots depict the distribution of each group, where the center of the x-axis denotes the point where the ALS FRS21-39 and healthy controls are maximally separated, and the y-axis reflects the count (or density estimate). The title of each plot indicates the clusters in the model. The accuracy of each model in indicated within the plot.

FIG. 20 shows results of a linear discriminant analysis: CD45+ Cells (Cluster>0.1%) Healthy (n=11), ALS FRS21-39 (n=17) and ALS FRS40-47 (n=11).

### DETAILED DESCRIPTION

The invention, in general terms, provides a method for defining stages and progression of Amyotrophic Lateral Sclerosis (ALS) and to inform clinical decision-making regarding diagnosis, therapeutic targeting and choice of therapy, treatment efficacy monitoring and prognosis.

Identification of immune signatures within clinical stages of ALS as described herein allows for development of targeted therapy and allows identification of patients for whom immune cell therapy is appropriate. This includes the application of specific B cell, T cell, NK cell, monocyte, dendritic cell, and mesenchymal cell therapeutic approaches that are personalized for each patient. The immune signatures disclosed herein and specific changes in the signature over time serve as multi parameter biomarkers of ALS progression or response to treatment (including anti-inflammatory/immune modulatory/neuro-immune modulatory approaches) and most importantly to guide clinical decision making. The immune signature of the ALS patient is readily applied for monitoring disease, making decisions regarding therapy application and response, predicting responses to therapy (in both standard of care and experimental care settings) as well as identifying a patient to target with therapy and prognostic markers of outcome that correlate with clinical biomarkers. In particular, a combined immune signature/clinical parameter (ALS-FRS score) may be delineated for these purposes.

The invention describes high dimensional immuno-phenotypic signatures and characteristics of patients with ALS that aid in the diagnosis and evaluation of disease progression and treatment. Immune profiling is achieved with single or multiple Omic (e.g., collective and high-throughput analyses including genomics, transcriptomics, proteomics, and metabolomics/lipidomics) technologies including but not exclusively flow cytometry, mass cytometry and single cell or total RNA sequencing in conjunction with clinical annotation of the clinical case. The immune signatures include definition of specific subpopulations of B cells, T cells, NK cells, monocytes, dendritic cells and neutrophils. In some embodiments, CyTOF methodology is employed with barcoding. Exemplary CyTOF methods are described in Zunder et al. (2015) Nature Protocols 10(2): 316 and Geanon et al. MedRxiv 10.1101/2020.06.26.20141341 (posted Jun. 29, 2020).

The methodology, in general, is as follows:
1. Draw a sample of peripheral blood into a tube containing anticoagulant from a patient with ALS and collect relevant clinical correlative information at the time of bleed including age, sex, date of onset of ALS, Revised Amyotrophic Lateral Sclerosis Functional Rating Scale-Revised (ALSFRS-R) score, hematological and other individual clinical chemical indices;
2. Separate white cells from the blood—e.g., by lysing the red cell component;
3. Perform, for example, mass or flow cytometry on the white blood cells using antibodies directed against:
   a. Canonical markers for granulocytes, monocytes, dendritic cells, T cells, B cells, and NK cells; and
   b. Activation markers that characterize both immune activating and immune suppressive cell subpopulations—including cytokines and chemokines; and
   c. Employ computational analysis to determine numbers of immune cells of each subpopulation or cluster and proportion of the total leukocyte population using a pan human leukocyte marker such as CD45;
4. Perform total RNA sequencing on samples to delineate both subpopulations of leukocyte populations based on gene expression as well as TCR and BCR expression analysis, viral genome analysis and HLA analysis (deep HLA classification); and
5. Perform computational analysis, including cluster analysis, linear regression analysis, linear discrimination analysis and elastic net logistical regression, in order to identify clusters of leukocytes in blood samples and those which differentially segregate between healthy individuals and individuals with ALS and between patient with early ALS (including ALS FRS scores—40-47 and 30-39).

In this scenario, if a profile contains a deficiency in regulatory or suppressive immune cell clusters (including regulatory T cells (Treg), regulatory B cells (Breg), and M2 macrophage clusters) and increased activated immune cell clusters T and B effector and NK effector cell clusters) the patient is classified as having ALS (along with concomitant clinical findings) the patient requires initiation of standard of care treatments for ALS such as Riluzole (Rilutek, Exservan, Tiglutik kit) and/or Edarvarone (Radicava), and/or sodium phenylbutyrate and taurursodiol (Relyvrio) using accepted dosing strategies for these agents. Treatment involving

If a profile taken sequentially from a patient shows a further increase in the deficiency of immune suppressive immune cell populations and increased activated immune cell clusters from an initial baseline recording in that patient along with concomitant decline in ALSFRS-R scores the patient should be considered for immune therapy including immune cell therapy (B cell or Treg cell) or immunotherapy with an appropriate immune modulating agent—including, for example, Baracitinib (Olumiant) or other Janus kinase/signal transducers and activators of transcription (Jak-Stat) inhibitors at a clinically approved dosing.

The methods and tests described herein can be used, e.g., individually or in combination with another clinical modality (e.g., ALS FRS-R) to improve and inform clinical decision-making regarding ALS diagnosis, therapeutic targeting and choice of therapy, treatment efficacy monitoring and prognosis.

### EXAMPLES

The following examples are put forth to provide those of ordinary skill in the art with a description of how the compositions and methods described herein can be used, made, and evaluated, and are intended to be purely exemplary of the disclosure and are not intended to limit the scope of what the inventors regard as their invention.

As used herein the term “sample,” when referring to the material to be tested for the presence of a biological marker using the method of the invention, includes inter alia whole blood, plasma, or serum. If needed, various methods are well known within the art for the identification and/or isolation and/or purification of a biological marker from a sample.

The relative level of each one of the cell types or subsets measured is represented in a profile by “increase,” indicating that the level of the cell type or subset in the blood sample obtained from the tested individual (e.g., a patient) is increased compared with the upper limit of the normal range level thereof, e.g., the range level of the cell type or subset in blood samples of controls, by at least about 10%, preferably at least about 20%, more preferably at least about 30%, 40%, or 50%; “decrease,” indicating that the level of the cell type or subset in the blood sample obtained from the tested individual is decreased compared with the lower limit of the normal range level thereof by at least about 10%, preferably at least about 20%, more preferably at least about 30%, 40%, or 50%; or “no change,” indicating that the level of the cell type or subset in the blood sample obtained from the tested individual is neither increased nor decreased as defined above, e.g., within or close to the normal range level thereof.

Although a reference profile according to the method of the present invention may be predetermined, it should be understood that this profile might be established using any suitable algorithm. For example, the representative relative level of a certain cell type or subset measured is represented by “increase,” indicating that the level of the cell type or subset in a majority of the ALS patients in the group is increased compared with the normal range level of the cell type or subset; “decrease,” indicating that the level of the cell type or subset in a majority of the ALS patients is decreased compared with the normal range level of the cell type or subset; or “no change,” indicating that the level of the cell type or subset in a majority of the ALS patients is neither increased nor decreased, as defined above, compared with the normal range level of the cell type or subset.

Suitable reference values can be determined using methods known in the art, e.g., using standard clinical trial methodology and statistical analysis. The reference values can have any relevant form. In some cases, the reference comprises a predetermined value for a meaningful level of a biomarker(s), e.g., a control reference level that represents a normal level of the biomarker(s), e.g., a level in an unaffected subject or a subject who is not at risk of developing ALS.

In some embodiments, a control subject is one that does not have ALS, does not have a risk of developing ALS, or does not later develop ALS. A control is, in general, a healthy subject.

An ALS subject is one who has (or has an increased risk of developing) ALS. An increased risk is defined as a risk above the risk of subjects in the general population.

The level of each one of the cell types or subsets disclosed herein, in the peripheral blood sample tested, can be measured utilizing any suitable technique known in the art.

### Example 1

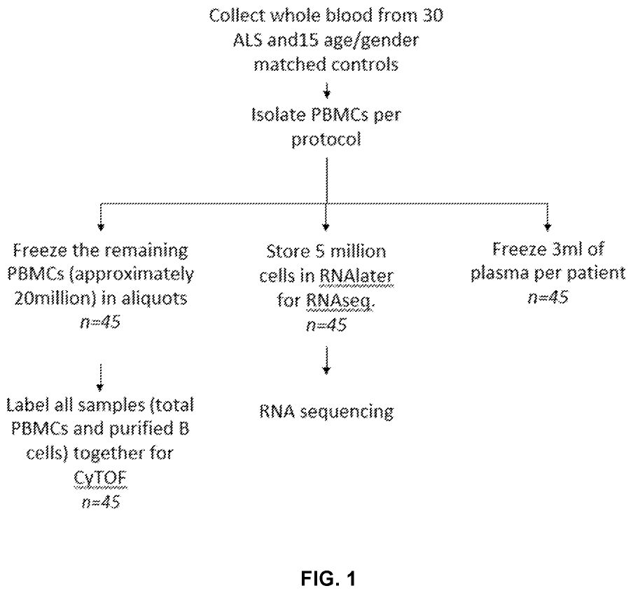

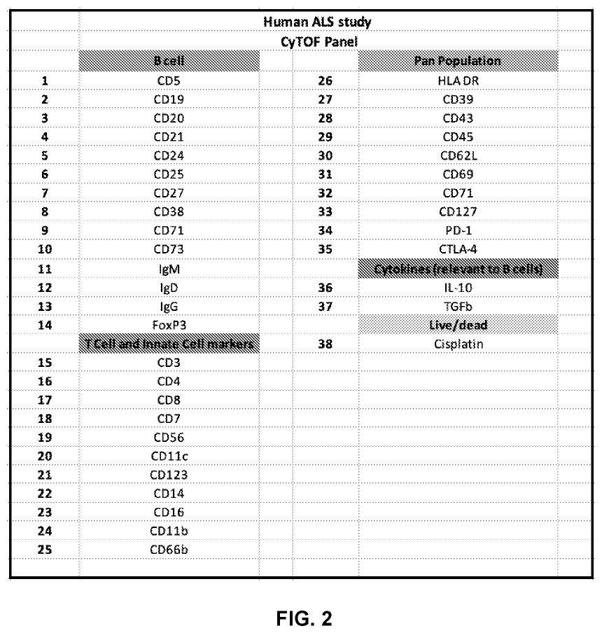

Mononuclear cells were isolated from blood samples of 30 individuals with ALS and 15 healthy controls collected over a period of one year (FIG. 1). A pilot study was conducted in which PBMC samples collected from 12 ALS patients and 6 age and gender matched healthy controls were labelled with metal conjugated antibodies and profiled by mass cytometry (CyTOF—Cytometry by time of flight) (FIG. 2).

The pilot study protocol was as follows:
Frozen PBMCs were thawed; 2 million live PBMCs were labelled for CyTOF analysis.
Each patient was surface labelled with a unique “Barcode”
   Barcodes are unique combinations of 3 metal tags. The metal conjugated antibodies used for the barcodes were:
      CD45 194Pt, CD45 195Pt, CD45 196Pt, CD45 198Pt, CD45 89Y;
      B2M 194Pt, B2M 195Pt, B2M 196Pt, B2M 198Pt, B2M 11Cd; and
      CD298 195Pt, CD298 196Pt, CD298 198Pt, CD298 112Cd.
After barcoding, samples were pooled; labelled with surface and intracellular markers; and data acquired on a Helios Cytometer according to standard methods.

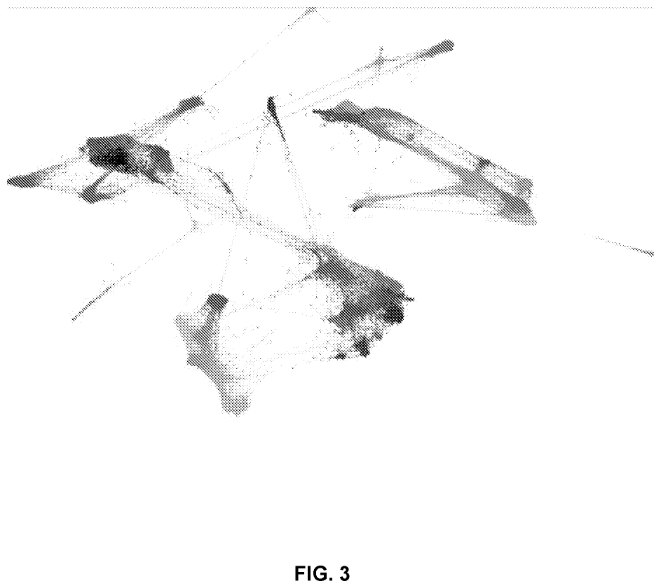

Data analysis (see, for example, FIG. 3) was conducted as follows:
Data was de-barcoded to identify each individual sample, using manual gating;
De-barcoded data was clustered using X-Shift
X-shift computes the density estimate for each data point. It then searches for the local density maxima in a nearest-neighbor graph, which become cluster centroids. All the remaining data points are then connected to the centroids via density-ascending paths in the graph, thus forming clusters.
A separate clustering was performed on B cells using X-Shift
# of phenotypic clusters in all CD45+ cells=52
# of phenotypic clusters in B cells=19

### Results:

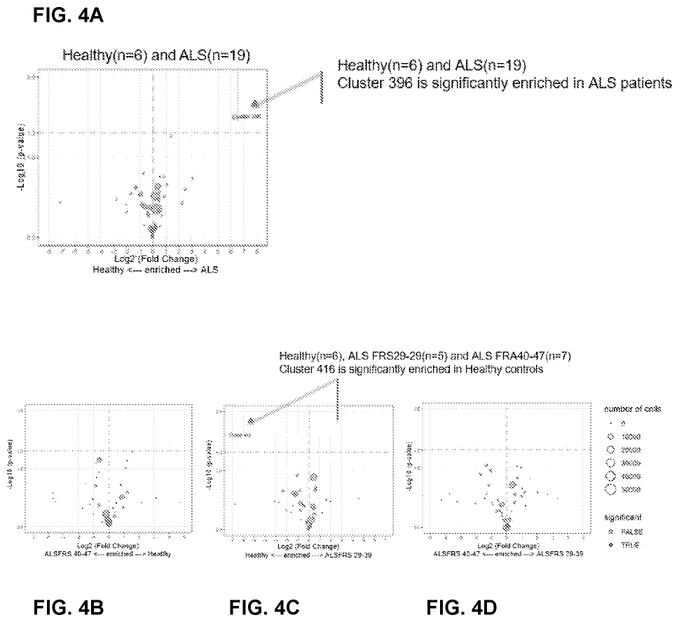

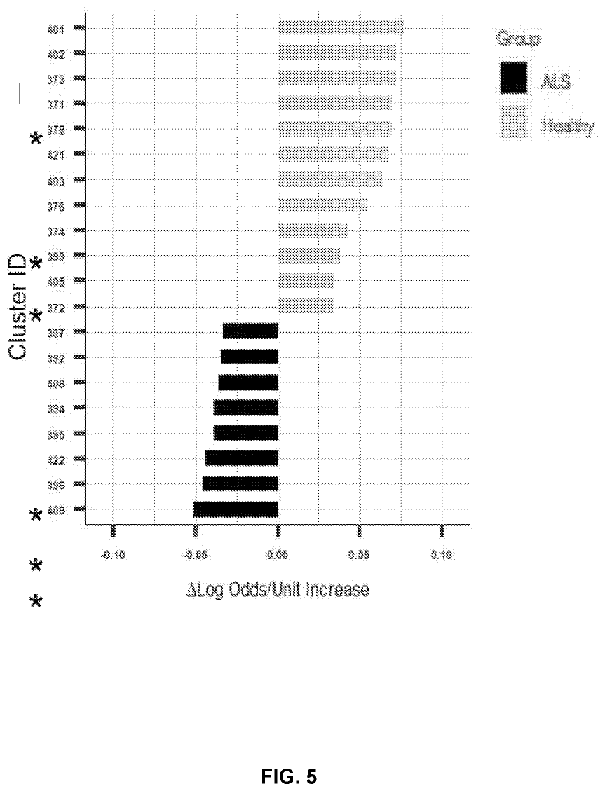

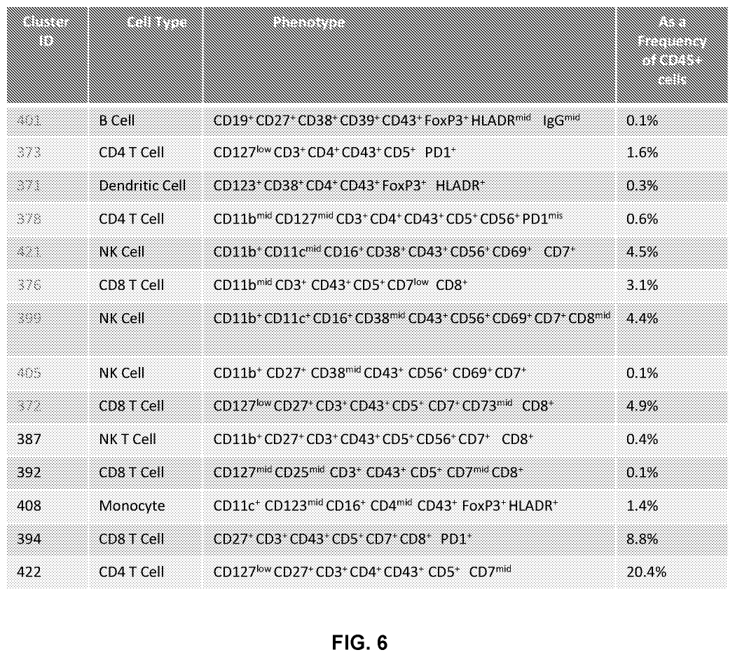

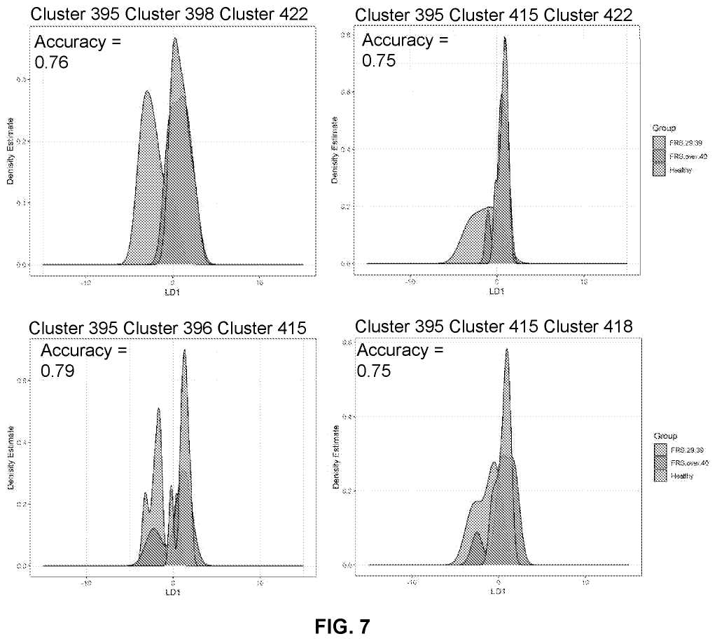

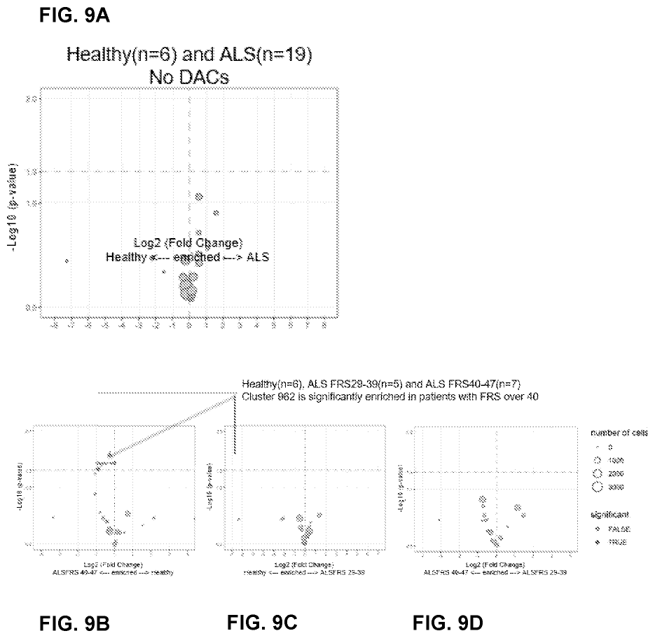

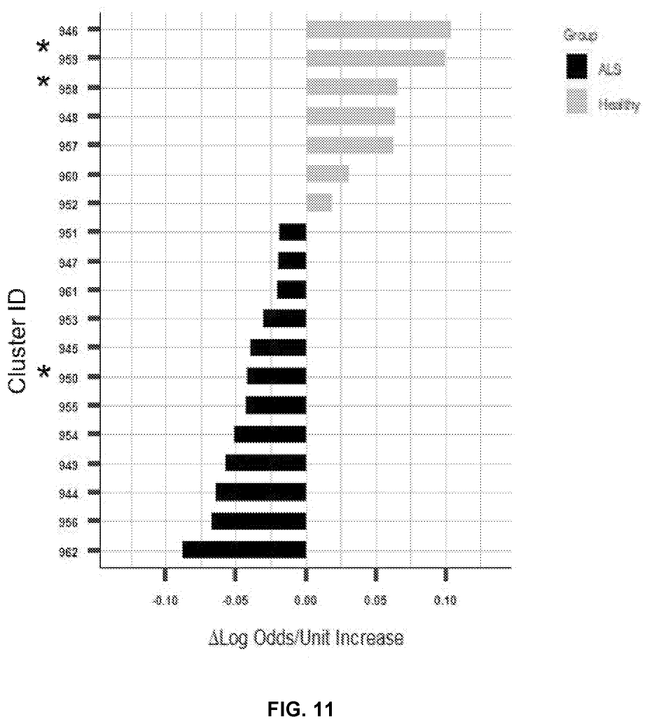

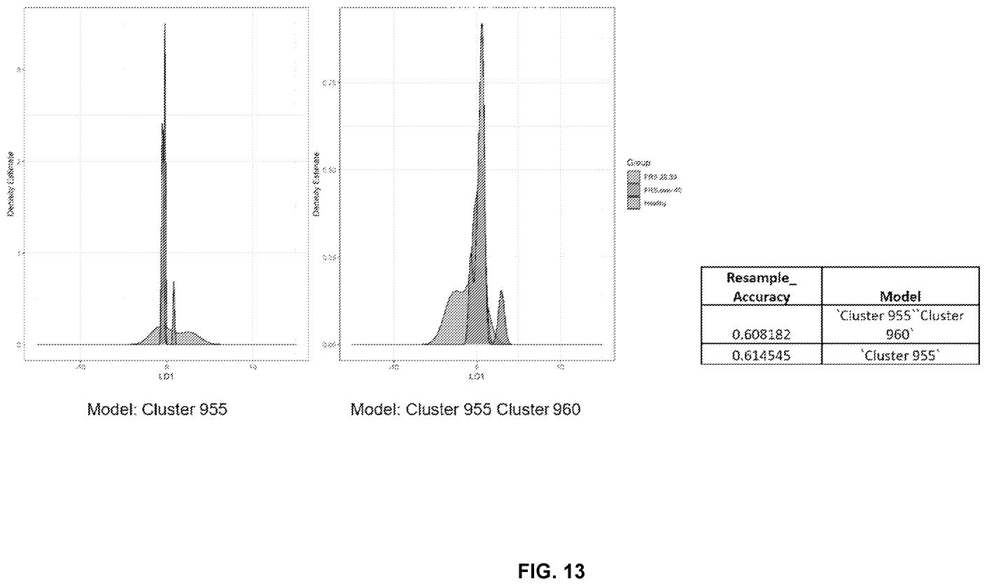

Results for Example 1 are described above and found in FIG. 3 through FIG. 15. These results are summarized as follows.

### A. B Cell Subpopulations of Interest:

Logistic regression model identified FoxP3+ B regulatory cells had a greater probability of having lower abundance in the peripheral blood of ALS patients. Linear Discriminant Analysis revealed that mature B cells (IgD+IgM+) with CD11c expression were increased in patients with lower ALSFRS-R as compared to higher ALSFRS-R and healthy controls (Accuracy of the model=0.60).

### B. T Cell Subpopulations of Interest:

Logistic regression model identified activated CD4 and CD8 T cell subtypes, as having a greater probability of higher abundance in the peripheral blood of ALS patients compared to healthy controls Linear Discriminant Analysis revealed that activated CD4 T cells were elevated in patients with lower ALSFRS-R as compared to patients with higher ALSFRS-R and healthy controls (Accuracy of the model=0.75)

### C. Innate Cell Subpopulations of Interest:

Logistic regression model identified CD11c+ monocytes, and NK T cells as having a greater probability of higher abundance in the peripheral blood of ALS patients compared to healthy controls.

Predictive models were utilized to identify clusters of interest due to the small number of patients studied and the large variation in immune profiles.

### Example 2

A second study was conducted of PBMC samples collected from 30 ALS patients and 15 age and gender matched healthy controls. Cells were labelled with metal-conjugated antibodies and profiled by mass cytometry (CyTOF). Mean age at the time of sample collection was 56.12 (Standard deviation=11.11). Mean ALS revised functional rating score (ALSFRS-R) of the ALS cohort was 36.5 (Standard deviation=6.6). Data acquired from CyTOF was clustered using k means.

The full CyTOF study protocol was as follows:
Frozen PBMCs were thawed; 2 million live PBMCs were labelled for CyTOF analysis.
Each patient was surface labelled with a unique “Barcode”
   Barcodes are unique combinations of 3 metal tags. The metal conjugated antibodies used for the barcodes are:
      CD45 194Pt, CD45 195Pt, CD45 196Pt, CD45 198Pt, CD45 89Y;
      B2M 194Pt, B2M 195Pt, B2M 196Pt, B2M 198Pt, B2M 11Cd; and
      CD298 195Pt, CD298 196Pt, CD298 198Pt, CD298 112Cd.
After barcoding, samples were pooled; labelled with surface and intracellular markers; data acquired on a Helios Cytometer.

Data analysis was conducted as follows:
Data was de-barcoded to identify each individual sample, using manual gating.
De-barcoded data was clustered using k means clustering.
   Approximately 92 clusters were identified with unique expression of the 37 CyTOF variables in the study
The cell clusters were assessed for differences between ALS patients and healthy controls through t-tests and logistic regression.

### Results:

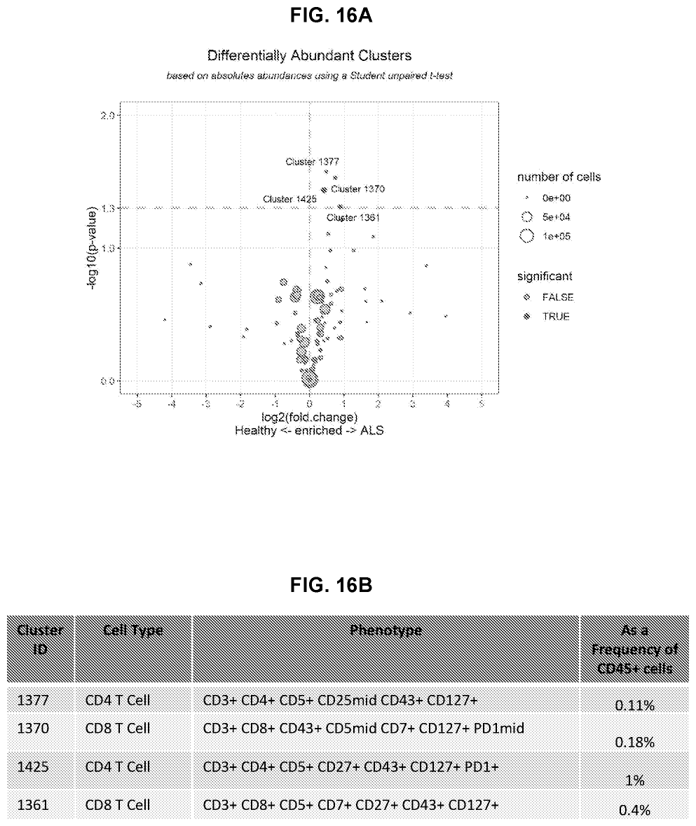

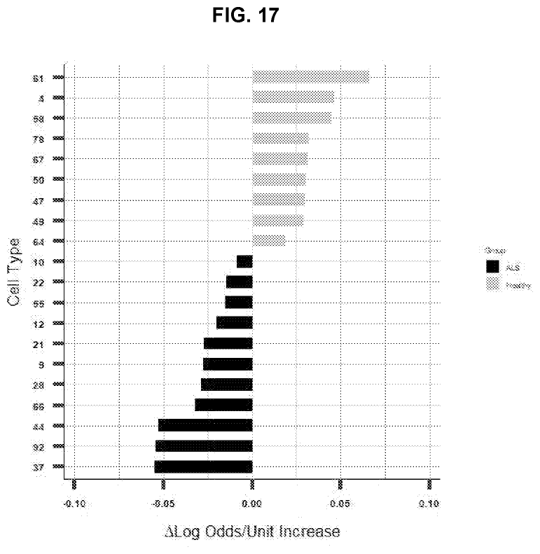

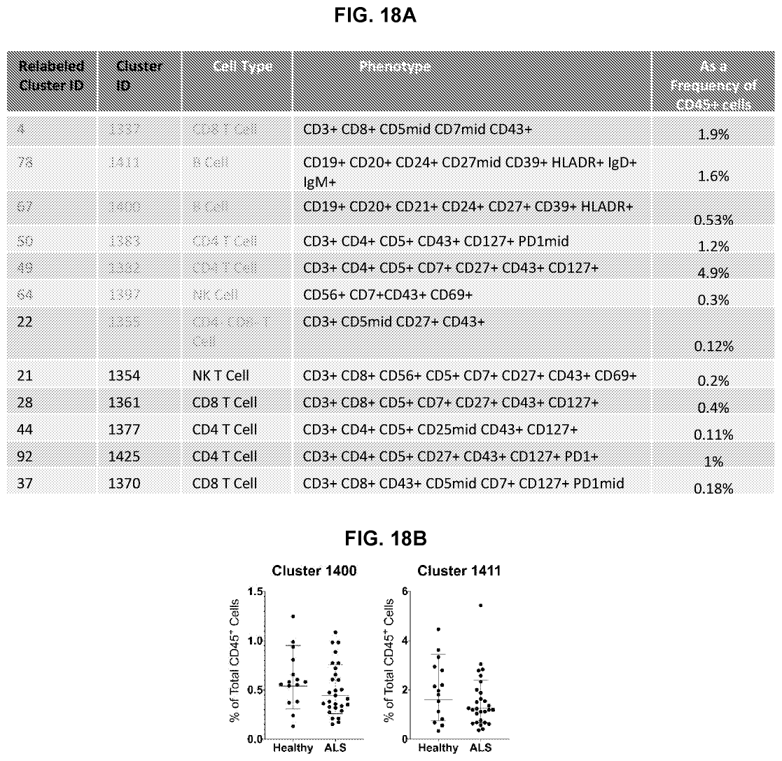

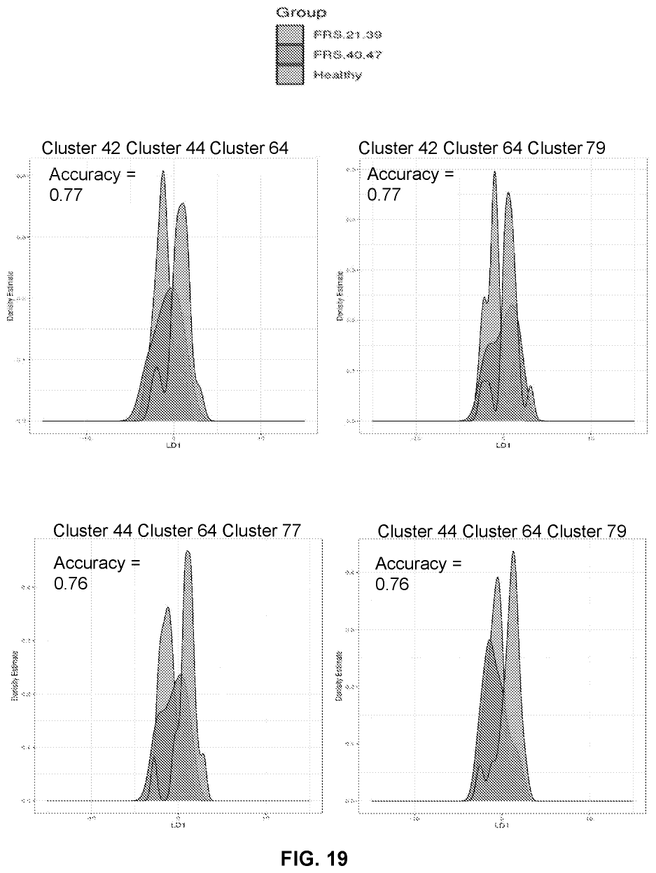

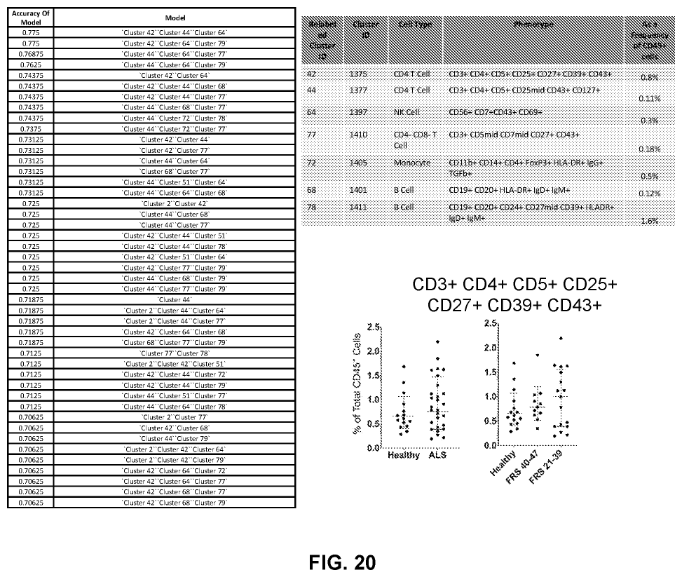

Results for Example 1 are described above and found in FIG. 16 through FIG. 20. These results are summarized as follows.

### A. B Cell Subpopulations of Interest:

Predictive models had to be utilized to identify clusters of interest due to the relatively small number of patients in the study. Activated B cells (CD19+ CD20+ IgD+IgM+) and memory B cells (CD19+ CD20+ CD21+ CD27+) had a greater probability of having lower abundance in the peripheral blood of ALS patients.

### B. T Cell Subpopulations of Interest:

Activated CD4 T cells (CD27+ PD1+) and CD8 T cell (CD27+ CD7+) populations are significantly increased in ALS patients as compared to healthy controls. Predictive models had to be utilized to identify clusters of interest due to the relatively small number of patients in the study. Within our training model, activated CD4 T cells (CD27+ PD1+) and CD8 T cell (CD27+ CD7+) populations were identified to have a greater probability of having higher abundance in the peripheral blood of ALS patients. Linear discriminant analysis identified a CD4 T cell population (CD25+ CD27+ CD39+) which is expressed more in patients with lower ALSFRS-R as compared to patients with higher ALSFRS-R and healthy controls (Accuracy=0.77)

### C. Innate Cell Subpopulations of Interest:

Preliminary logistic regression model identified only NK T cells as having a greater probability of higher abundance in the peripheral blood of ALS patients compared to healthy controls.

The findings of the full study (Example 2) are consistent with the results generated during the pilot study (Example 1). These specifically identified immune subpopulation differences represent a clinical decision-making tool that would help indicate when to initiate standard of care and what specific therapy (anti-inflammatory/immune modulatory or other) and when to intervene with these therapies during the course of the patient's disease. This would be the first biomarker signature that could be used as a clinical decision-making tool that looks well beyond a single chemical biomarker of disease progression in ALS, which has been elusive up until now.

The immune signatures of the invention can be used in several ways including:
1. Monitoring. The immune profile changes over the course of the disease. Correlation analysis between the immune phenotype and ALSFRS-R may indicate the appropriate timing of treatment with immune modulatory drug therapy or immune cell therapy.
2. Predictive biomarkers. The immune signature profiling guides timing and patient selection for any given immune therapy.
3. Prognostic. Prognostic biomarkers predicts disease outcome and tend to stay the same over the course of the disease. For example, neurofilament is elevated in rapid progressors. It goes up early and stays there. This means it is NOT a good monitoring biomarker. Immune profiling yields flexible signatures that can work as disease monitoring biomarkers. To date we have demonstrated a difference between the immune profile of patients with early and late ALS based on ALSFRS-R scores.
4. PD/drug response—the immune signature could provide a good pharmacodynamic (PD)/response biomarker for a clinical trial. This is closely connected with being a monitoring biomarker. It could be a PD biomarker for an immune therapy even if it does a poor job of monitoring disease. This is in marked comparison with state of the art subjective and qualitative clinical function scores like the ALS-FRS score or individual biochemical indices including neurofilament measurements in the serum or spinal fluid.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

Other embodiments are within the following numbered paragraphs.
1. A method of diagnosing ALS in a patient comprising:
   (a) on a sample of white cells obtained from the peripheral blood of the patient, performing mass cytometry on the sample using antibodies directed against (i) Canonical markers for granulocytes, monocytes, dendritic cells, T cells, B cells, and NK cells; and (ii) Activation markers that characterize both immune activating and immune suppressive cell subpopulations—including cytokines and chemokines;
   (b) applying computational analysis to determine numbers of immune cells of each subpopulation or cluster and proportion of the total leukocyte population using the pan human leukocyte marker CD45;
   (c) performing total RNA seq on the patient sample to delineate both subpopulations of leukocyte populations based on gene expression as well as TCR and BCR expression analysis, viral genome analysis and HLA analysis (deep HLA classification); and
   (d) analysis and elastic net logistical regression to identify clusters of leukocytes in the patient sample and those clusters of specific subpopulations of immune and inflammatory cell populations which differentially segregate between healthy individuals and individuals with early or late ALS based on FRS-R scores—wherein:
   (I) If the patient sample contains a deficiency in regulatory or suppressive immune cell clusters, including Treg, Breg, M2 macrophage clusters, and increased activated immune cell clusters T and B effector and NK effector cell clusters, the patient is considered to have ALS and requires initiation of Riluzole and/or Edarvarone using accepted dosing strategies for these agents, and if the patient sample does not contain the deficiency and the increase activated immune cell clusters the patient is unlikely to have ALS and no medication is required.
2. A method of assessing the progression of ALS in a patient and determining appropriate treatment comprising:
   (a) using the method of claim 1 to determine whether the patient has ALS;
   (b) obtaining sequential samples of white cells from the peripheral blood of the patient and subjecting those samples to the method of claim 1, wherein if the profile taken sequentially from a patient shows a further increased in the deficiency of immune suppressive immune cell populations and increased activated immune cell clusters from an initial baseline recording in that patient along with concomitant decline in ALSFRS-R scores, consider the patient for immune therapy including immune cell therapy (B cell or Treg cell) or immunotherapy with immune modulating agents—including for example Baracitinib or other Jak-Stat inhibitors or other repurposed or novel drugs or cell therapies being trialed for use in ALS at clinically approved dosing.

What is claimed is:

1. A method comprising characterizing a white blood cell sample from a patient using cytometry; wherein a deficiency in regulatory or suppressive immune cells and increased activated immune cells in the sample, relative to a healthy sample, indicates that the patient has amyotrophic lateral sclerosis (ALS).

2. The method of claim 1, wherein the sample is incubated with antibodies that specifically bind granulocytes, monocytes, dendritic cells, T cells, B cells, NK cells, immune activating cells, or immune suppressive cells.

3. The method of claim 1, wherein the regulatory or suppressive immune cells comprise Treg, Breg, or M2 macrophage clusters.

4. The method of claim 1, wherein the activated immune cells comprise T and B effector and NK effector cell clusters.

5. The method of claim 2, comprising calculating numbers of immune cells and proportion of the total leukocyte population of the sample using a pan human leukocyte marker.

6. The method of claim 5, wherein the pan human leukocyte marker is CD45.

7. The method of claim 1, wherein cytometry is cell or mass cytometry.

8. The method of claim 1, comprising performing total RNA sequencing on the sample to delineate subpopulations of leukocyte populations and TCR and BCR expression analysis, viral genome analysis and/or HLA analysis.

9. The method of claim 1, comprising identifying clusters of leukocytes in the sample.

10. The method of claim 9, wherein identifying comprises cluster analysis, linear regression analysis, linear discrimination analysis and/or elastic net logistical analysis.

11. The method of claim 10, wherein clusters of leukocytes segregate between healthy individuals and individuals with ALS.

12. The method of claim 11, wherein the ALS is late or early ALS.

13. The method of claim 1, wherein the patient has a deficiency in Treg, Breg, or M2 macrophage clusters.

14. The method of claim 1, wherein the patient has increased activated immune cell cluster T and B effectors.

15. The method of claim 1, wherein the patient has increased activated NK effector clusters.

16. The method of claim 1, further comprising administering to the patient a therapy for treating ALS.

17. The method of claim 16, wherein the therapy is riluzole or edarvarone.

18. The method of claim 1, wherein FoxP3+ B regulatory cells have lower abundance in the ALS patient.

19. The method of claim 1, wherein mature B cells comprising CD11c expression are increased in patients with a lower ALSFRS-R as compared to higher ALSFRS-R and healthy controls.

20. The method of claim 1, wherein CD4 T cells are increased in the ALS patient.

21. The method of claim 1, wherein CD8 T cells are increased in the ALS patient.

22. The method of claim 1, wherein activated CD4 T cells are elevated in an ALS patient having a lower ALSFRS-R as compared to patients with higher ALSFRS-R and healthy controls.

23. The method of claim 1, wherein CD11c+ monocytes are increased in the ALS patient.

24. The method of claim 1, wherein NK T cells are increased in the ALS patient.

25. The method of claim 1, wherein activated B cells (CD19+ CD20+ IgD+ IgM+) are decreased in the ALS patient.

26. The method of claim 1, wherein memory B cells (CD19+ CD20+ CD21+ CD27+) are decreased in the ALS patient.

27. The method of claim 1, wherein activated CD4 T cells (CD27+ PD1+) are increased in the ALS patient.

28. The method of claim 1, wherein CD8 T cells (CD27+ CD7+) are increased in the ALS patient.

29. The method of claim 1, wherein CD4 T cells (CD25+ CD27+ CD39+) are increased in ALS patients with lower ALSFRS-R as compared to ALS patients with higher ALSFRS-R and healthy controls.

30. The method of claim 1, wherein NK T cells are increased in ALS patients.

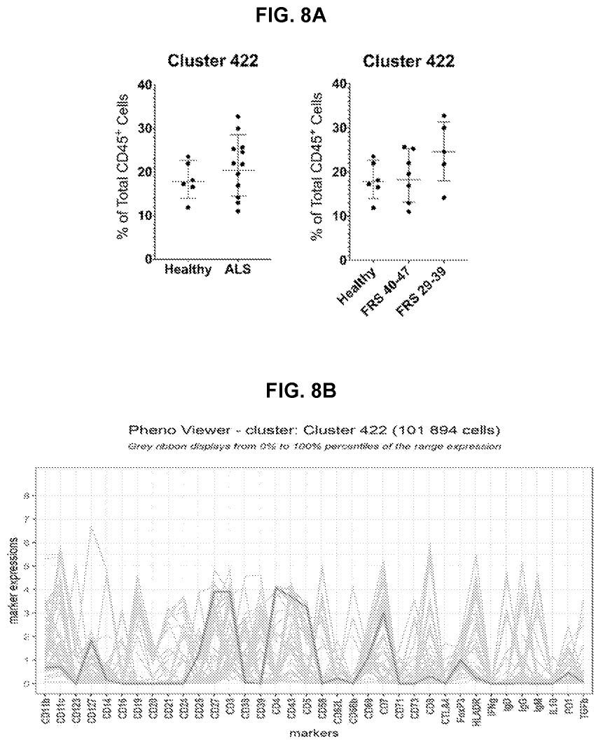

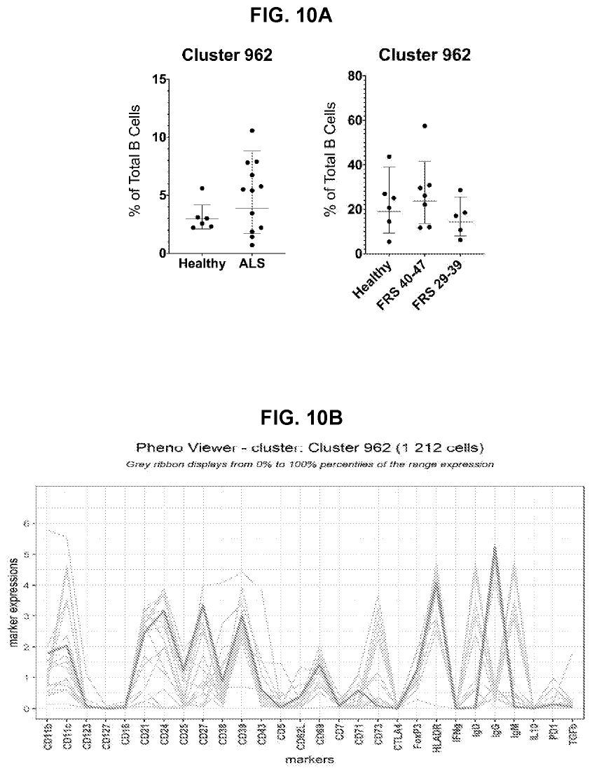

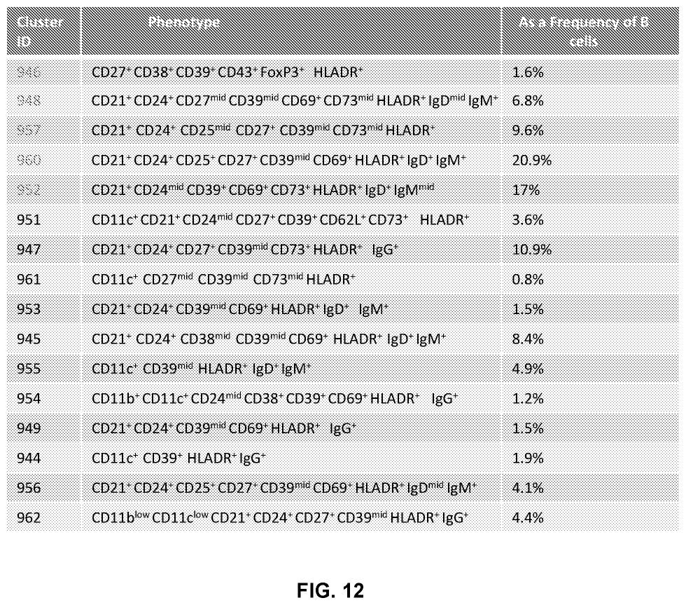

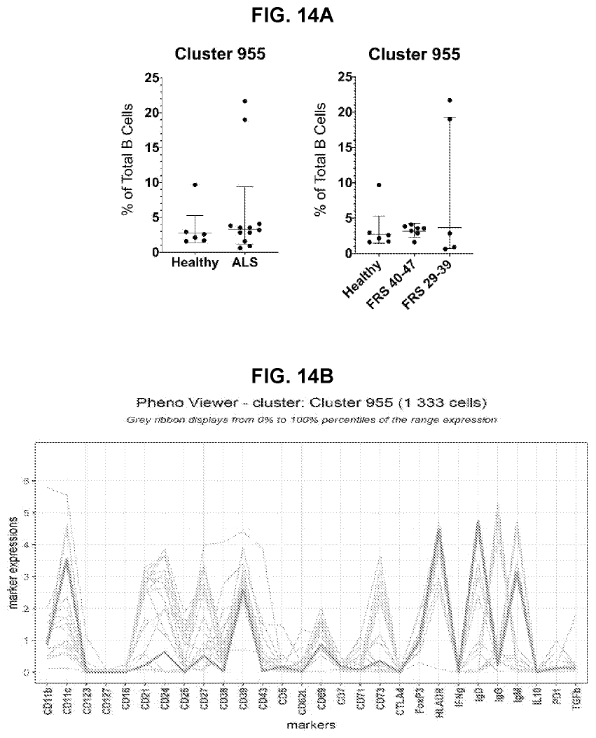

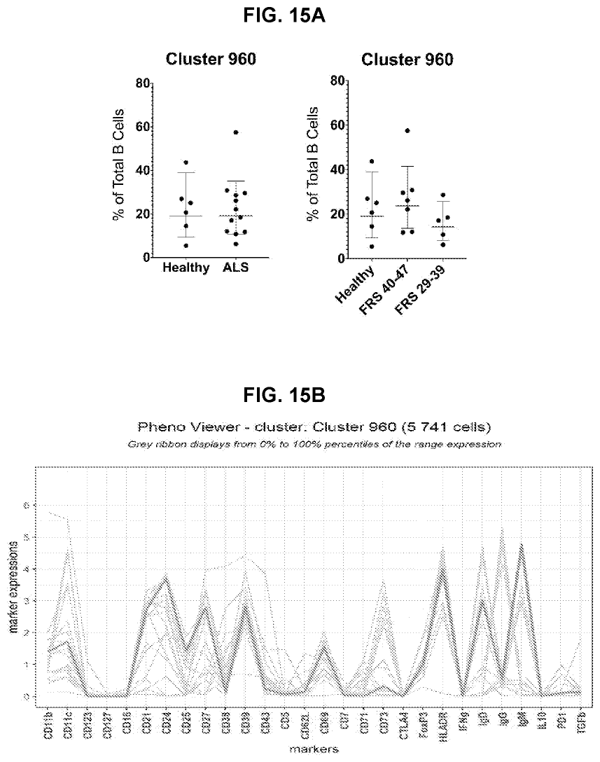

31. The method of claim 1, comprising determining a level of at least 2, 3, 4, 5, 6, 7, 8, 9, 10, 11, 12 or more markers as listed in FIG. 2, FIG. 8B, FIG. 10B, FIG. 12, FIG. 14B, and FIG. 15B.

32. The method of claim 1, wherein the sample comprises a phenotype as depicted in cluster 387, 392, 394, 408, or 422.

33. The method of claim 1, wherein the sample comprises a phenotype as depicted in cluster 951, 947, 961, 953, 945, 955, 954, 949, 944, 956, or 962.

34. The method of claim 1, wherein the sample comprises a phenotype as depicted in cluster 21, 28, 44, 92, or 37.

35. A method comprising:
(a) determining whether a patient has ALS according to any of the aforementioned claims;
(b) analyzing a second white blood cell sample from the patient according to any of the aforementioned claims; and
(c) determining a deficiency in regulatory or suppressive immune cells and increased activated immune cells in the second sample.

36. The method of claim 35, the patient has an increased deficiency in Treg, Breg, or M2 macrophage clusters in the second sample compared to the sample of claim 1.

37. The method of claim 35, wherein the patient has increased activated immune cell cluster T and B effectors in the second sample compared to the sample of claim 1.

38. The method of claim 35, wherein the patient has increased activated NK effector clusters in the second sample compared to the sample of claim 1.

39. The method of claim 35, further comprising administering to the patient a therapy for treating ALS.

40. The method of claim 39, wherein the therapy comprises immune cell therapy.

41. The method of claim 40, wherein immune therapy comprises administering B cells or Treg cells.

42. The method of claim 39, wherein the therapy comprises administering an immune modulating agent.

43. The method of claim 42, wherein the immune modulating agent is Baracitinib or a Jak-Stat inhibitor.

44. The method of claim 35, wherein the patient is experiencing a clinically meaningful decline from baseline in an ALSFRS-R total score at the time the second sample is obtained.

45. The method of claim 35, wherein there are at least 2, 4, 6, 8, 10, 12, or 14 weeks between obtaining the sample of claim 1 and the second sample.

46. The method of any one of claims 1-45, wherein the patient is human.

47. A method of treating ALS, said method comprising performing flow cytometry using a blood sample obtained from a human according to the methods of claims 1-15 and 18-35 to identify the human as having ALS and administering an ALS therapy to said human.

48. A method of treating ALS, said method comprising administering a therapy to an ALS patient identified as having ALS according to the methods of claims 1-15 and 18-35.

49. The method of claim 47, wherein ALS therapy involves administering riluzole or edavarone.

50. The method of claim 48, wherein therapy comprises immune cell therapy.

51. The method of claim 48, wherein therapy comprises administering an immune modulating agent.
